(19) United States
(12) Patent Application Publication
(10) Pub. No.: US 2009/0082659 A1
(43) Pub. Date: Mar. 26, 2009

(54) MEDICAL EXAMINATION APPARATUS

(75) Inventors: Cornelis Leonardus Gerardus Ham, Eindhoven (NL); Frederik Anthonie De Graaf, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/719,101

(22) PCT Filed: Nov. 10, 2005
(86) PCT No.: PCT/IB2005/053698

§ 371 Date: May 11, 2007

(30) Foreign Application Priority Data
Nov. 12, 2004 (EP) 04105728.2

(51) Int. Cl. A61B 5/055

(52) U.S. Cl. 600/411

(57) ABSTRACT

The invention relates to a medical examination apparatus, in particular an MR apparatus. Known MR apparatus have a cavity for accommodating the patient to be examined that offers only a limited space for the patient. The limited space may lead to claustrophobic reactions jeopardizing the result of a measurement. According to the invention a mirror (4), arranged in parallel to the main surface (5) of the patient's bed (3), is provided in the interior of the cavity (1). The mirror (4) simulates a larger space of the interior of the cavity and increases the patient's comfort.

The invention relates to a medical examination apparatus, comprising a cavity where an object or a patient is subjectable to electromagnetic waves, and a patient bed within the cavity. In particular it relates to a medical examination apparatus for use in MR (magnetic resonance) systems, MRI (Magnetic resonance imaging) systems, CT (computer tomography) systems, or PET-scanners.

MR systems comprise an at least partially closed cavity where a patient, or a part of the patient's body, is located. A static magnetic field is generated which fills the measuring space defined by the cavity. Signals of an RF (radio frequency) transmitter are coupled into the body which lift a magnetic degeneracy of selected nuclei within the body. The nuclei absorb RF energy and dissipate this energy within a relaxation time. Studying the 3D (three dimensional) distribution of relaxation times makes it possible to obtain 3D images of tissue of the patient.

For accurate 3D images of the tissue the patient is not allowed to move. However, the limited space offered in the cavity makes many patients feel uncomfortable or even anxious. Claustrophobia makes the patients move or even leave the MR cavity. Similar considerations apply to CT systems.

US 2003/0128034 A1 discloses a medical examination apparatus of tubular shape. Inside the tubular apparatus is a stereoscopic image carrier that simulates that a larger space is arranged in the examination space in the field of the patient. The larger space simulated by the stereoscopic images, for example holograms, should help to prevent claustrophobic anxieties.

WO 01/22108 A1 discloses a magnetic resonance apparatus which uses a mirror within the cavity. The surface of the mirror inclines with respect to the patient's bed. The mirror enables the patient to maintain visual contact with the outside world.

It is an object of the invention to provide a medical examination apparatus of the kind mentioned in the opening paragraph with a cavity that simulates to be larger in space than it really is.

In order to achieve said object, a medical examination apparatus according to the present invention is characterized in that the apparatus further comprises a substantially flat mirror arranged in parallel to a main surface of the patient's bed.

In the following the description will only refer to the case in which a person or an animal and thus a “patient” is examined in the cavity. The invention however is not restricted to this case, as the man skilled in the art will easily understand that it is also possible to examine objects in the cavity such as plants or other non-living material.

The invention rests on the idea that the patient's perception of the space of the cavity will be influenced when a flat mirror is arranged above the patient's face in parallel to the main surface of the patient's bed. The main surface of the patient's bed shall be defined to be the surface on which the patient rests during the examination. In this case the patient can look at himself and can see the interior of the cavity more easily. Depending on the size of the mirror the patient can even see the whole cavity from within. The patient's impression is that the patient's space is increased by a factor of 2 or more in comparison to a situation in which there is no mirror within the cavity. The perceived space, which shall be defined to be space the patient perceives when he or she is within the cavity, seems to be larger than without a mirror. As a consequence, the patient's comfort and his acceptance of the apparatus is improved.

The preferred embodiments of the invention relate to a MR apparatus. The MR apparatus can be of conventional type with a closed cylindrical cavity, wherein the cylindrical cavity has two openings being located in a plane perpendicular to the longitudinal axis of the cavity. The most preferred embodiments are open MR systems where the cavity, in addition to the above mentioned openings, is open on the left and the right of the patient when he or she lies on the patient's bed. The MR apparatus generates magnetic fields up to about 3 T. The frequency of the RF field is 42 MHz if the field is 1 T, and the frequency is proportional to the magnetic field strength.

The mirror may be provided at the inner walls of the patient's space which form the cover of the magnet. In particular, the mirror can be integrated into the covers.

To increase the perceived patient's space the surface of the mirror is preferably chosen to be larger than the surface of an adult's face, and preferably larger than about 26 cm in diameter.

The shape of the mirror may be adapted to the geometry of the cavity. If a conventional MR apparatus is chosen the mirror may extend along a substantial part of the longitudinal axis of the cylindrical cavity. A substantial part shall be defined to be at least one third of the length of the patient's space when measured in the direction of the longitudinal axis. The mirror may extend along one third, one half, or even two thirds of this length, and the mirror may be rectangular. If the longitudinal axis is estimated to be 2 meters long, the extension of the mirror in this direction would be 66 cm, 1 m, or even 1.3 m. Good results were achieved with rectangular shapes of 1 m×26 cm in size.

If an open MR apparatus is chosen a circular mirror can be chosen. The diameter is at least about 80 cm, preferably at least about 1 m.

The mirror is chosen to be a substantially flat mirror as a curved mirror results in a distortion in the visual perception of the cavity. In this respect a non-distorting flat mirror is more comfortable and avoids hilarious reactions.

In this respect an alignment of a flat mirror parallel to the patient's bed makes it possible to place the mirror relatively far away from the patient's face, in particular in comparison to a situation where the mirror is at an angle with respect to the main surface of the patient's bed. In the latter case the distance of the mirror from the eye of the patient is roughly 10 cm, in the first case 15 cm is possible. It should be born in mind that the minimum distance the eye can accommodate an object for a longer time is normally 25 cm. This means that in the case of a mirror being aligned in parallel to the patients bed the patient can look at his own face without any efforts.

The mirror should disturb the functioning of the apparatus as little as possible. However, mirrors reflecting light in the visible part of the electromagnetic spectrum normally contain a layer consisting of a material with a high reflection coefficient such as silver or aluminium. If a RF field is coupled into these conductive layers eddy currents are generated. This is a particular concern for larger mirrors.

One possibility to avoid these eddy currents are metal-free mirrors, such as mirrors having a multitude of dielectric coatings. Another possibility would be a very thin metallic layer in the sense that the thickness of the layer is much smaller than the skin depth of the RF waves uses in the apparatus. As a general rule, the thickness of the layer should be one order of magnitude smaller than the skin depth. Typically this means a thickness of a few micrometers.

A preferred embodiment uses a mirror which comprises a layer which consists of a multitude of metallic areas. The areas can be of arbitrary shape and might be dots or strips which have no galvanic connection with each other. An insulator might be between the above mentioned areas. The lack of a galvanic connection results in a decreased electric path length which makes the creation of eddy currents more difficult. Although this type of mirror shows a relatively low reflection coefficient, the result is satisfactory particularly when the light intensity in the patient's space is relatively low.

Another preferred embodiment uses a mirror with a metallic layer, wherein the layer comprises recesses such as slits. This results in an increased electric path length which effectively avoids eddy currents.

The size of the metallic areas mentioned in the penultimate paragraph, or the size of the recesses mentioned in the last paragraph, depends on the local RF field strength at the mirror as transmitted by the transmit coil, and depends on the thickness of the layer in relation to the skin depth. Additional recesses might be needed to reduce eddy currents which are induced by switching the gradient coil.

Embodiments of a medical examination apparatus in accordance with the invention will be described in detail in the following with reference to the drawings, in which:

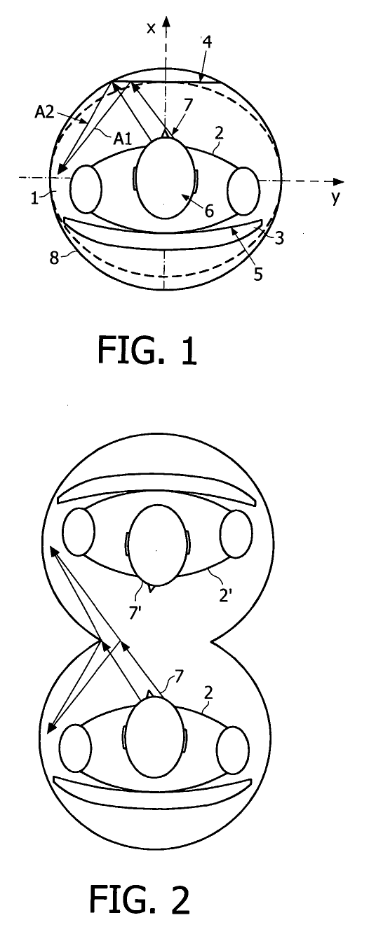

FIG. 1 shows a cavity of a closed cylindrical MR apparatus according to the invention when viewed in a direction of a symmetry axis of the cavity;

FIG. 2 shows the experienced spacious view in the cavity of FIG. 1;

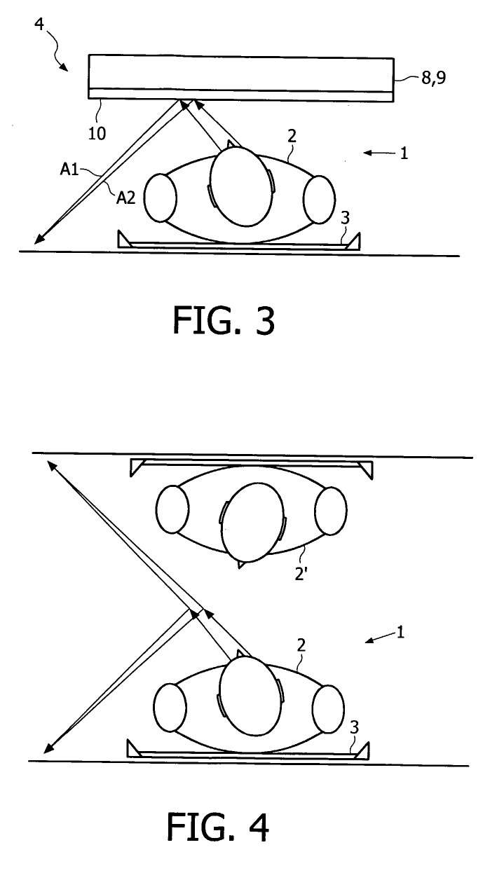

FIG. 3 shows a cavity of an open MR apparatus according to the invention when viewed in a direction of a symmetry axis of the cavity;

FIG. 4 shows the experienced spacious view in the cavity of FIG. 3;

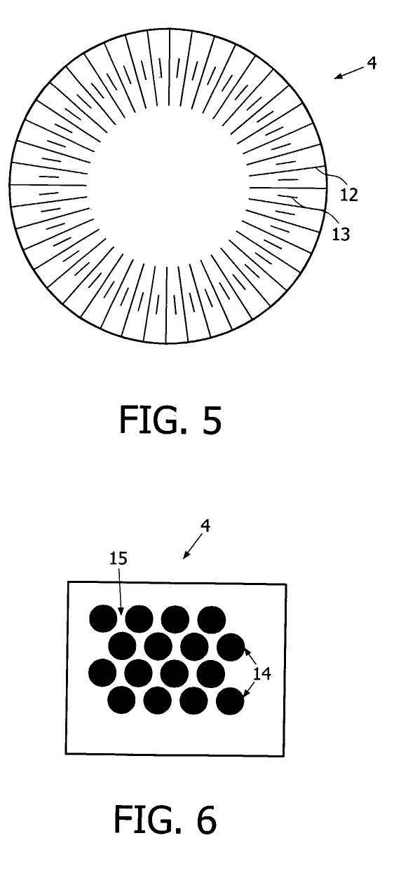

FIG. 5 shows an embodiment of a mirror used in an MR apparatus according to the invention; and

FIG. 6 shows an alternative mirror having a layer with metallic dots.

FIG. 1 shows a cavity 1 of a closed cylindrical MR apparatus according to the invention. The view is along a longitudinal axis (z-axis, axis of symmetry) which is both perpendicular to the x-axis and the y-axis. The x-axis, the y-axis and the z-axis represent a 3D coordinate system. The cavity has an inner diameter of 60 cm and has two openings which both lie in the xy-plane.

Inside the cavity is a patient 2 lying on a patient's bed 3. Although the patient's bed 3 has a partially curved main surface 5, its central part is approximately flat and lies in the zy-plane. The patient looks in the x-direction which is normal to the main surface 5 of the patient's bed 3. Above his head 6 is a flat mirror 4 arranged in the zy-plane and thus in parallel to the main surface 5 of the patient's bed 3. The distance of the mirror to the center of the coordinate system is 27 cm. The center of the mirror 4 has a distance of 3 cm to the upper cover 8.

The mirror is integrated into the cover, such that the cover is flattened in this region. In the alternative, the mirror can be placed inside the QBC (quadrature body coil), wherein the QBC cover 8 is chosen to be transparent. In this case the distance of the mirror 4 to the patient's bed 3 can be increased and the mirror 4 can be made larger. In the latter case the perceived space is increased even more.

As an alternative, the QBC (and thus the cavity) of the MR apparatus doesn't have a circular shape within the xy-plane, but has an oval shape. This is indicated by the dotted line.

The perception the patient 2 has when he lies on the patient's bed 3 is indicated by the arrows A1, A2. When looking above the patient can see himself or can look to the left and right. The experienced space is like looking through a window as indicated by FIG. 2. The impression is that the patient's face 7 is not 15 cm away from the mirror, but is 30 cm away from his mirror image 2′. Correspondingly, the patient 2 perceives a space between himself and his mirror image 2′ which is larger than without the mirror 4.

FIG. 3 shows an Open MR system with a flat cover 8 on top of a QBC (not shown). The cover 8 is 2 mm thick and is made of polycarbonate. The cover 8 acts as a substrate 9 for metal-free and non-conducting reflection layer 10. Cover 8 and layer 10 are not to scale in order to visualize the composition of the circular mirror 4 which has a diameter of 1 m.

FIG. 4 shows the perception the patient 2 has when he lies on the patient's bed 3 on the MR apparatus of FIG. 3. Similar to the situation of FIG. 2 the perceived space is larger than the real space because the distance of the patient 2 to his mirror image 2′ is larger than to the mirror 4.

FIG. 5 shows an embodiment of a mirror usable in an Open MR apparatus. The mirror 4 has a diameter of 1 m and contains a layer of aluminium having a thickness of 0.1 m. The mirror 4 has an alternating sequence of long slits 12 and short slits 13. All slits 12, 13 are arranged in a radial direction which increases the electrical path length. The smaller slits 13 are located in a region where the RF field strength is particularly high.

FIG. 6 shows a mirror 4 which comprises a layer 10 which consists of a multitude of metallic areas 14. The galvanic areas 14 are small dots, wherein the region 15 does not contain a metal and thus prevents an electric connection between the metallic areas 14. The size of the dots are not to scale for illustration purposes.

1. A medical examination apparatus, comprising a cavity where an object or a patient subjectable to electromagnetic waves, and a patient bed within the cavity, wherein the apparatus further comprises a substantially flat mirror arranged in parallel to a main surface of the patient's bed.

2. An apparatus according to claim 1, wherein a surface of the mirror is larger than a surface of an adult patient's face.

3. An apparatus according to claim 1, wherein the apparatus is a closed cylindrical MR apparatus, wherein the mirror extends along a substantial part of a longitudinal axis of the cavity.

4. An apparatus according to claim 1, wherein the apparatus is an open MR apparatus, wherein the mirror has a diameter of at least 80 cm.

5. An apparatus according to claim 1, wherein the mirror is metal-free.

6. An apparatus according to claim 1, wherein the mirror comprises a metal layer having a thickness much smaller than a skin depth corresponding to a frequency of the electromagnetic waves the patient is subjectable to.

7. An apparatus according to claim 1, wherein the mirror comprises a layer which consists of a multitude of metallic areas with no galvanic connection between them.

8. An apparatus according to claim 1, wherein the mirror comprises a metallic layer with recesses.

9. An apparatus according to claim 1, wherein the apparatus is a magnetic resonance system, and particularly is a magnetic resonance imaging system.

10. An apparatus according to claim 1, wherein the apparatus is an open magnetic resonance system, particularly an open magnetic resonance imaging system

11. An apparatus according to claim 1, wherein the apparatus is a computer tomography system or a PET scanner.
